(19) United States
(12) Patent Application Publication
(10) Pub. No.: US 2010/0323387 A1
(43) Pub. Date: Dec. 23, 2010

(54) Optimization of Response to Light

(76) Inventors: Shaun Bailey, Los Altos, CA (US); Bertrand Vick, Emeryville, CA (US)

Correspondence Address: CARR & FERRELL LLP, 120 CONSTITUTION DRIVE, MENLO PARK, CA, 94025, US

(21) Appl. No.: 12/488,106

(22) Filed: Jun. 19, 2009

(51) Int. Cl. C12Q 1/02; C12M 1/34

(52) U.S. Cl. 435/29; 435/288.7

(57) ABSTRACT

Various aspects provide for exposing a substance to light. Certain aspects include exposing a suspension of photosynthetic organisms to sunlight, and may include optimizing exposure to improve photosynthesis conditions. Certain embodiments include controlling an opacity or opacity profile of a suspension of algae and/or diatoms. Optimizing exposure may include maximizing growth rate, maximizing photosynthesis efficiency, maximizing lipid production, minimizing damage, minimizing predator growth, maximizing a capacity to grow in suboptimal media (e.g., polluted water, brackish water, or water having a pH outside of a preferable range), minimizing requirements for nutrients, and other features.

### BACKGROUND

1. Technical Field

This application relates generally to exposing substances to light, and more particularly to optimizing photosynthesis of suspended organisms.

2. Description of Related Art

Many processes entail exposing a material to light (e.g., sunlight). An exposed material may be at least somewhat transparent, and may scatter or absorb light through a volume (or depth). Transparency may be characterized in the inverse (e.g., by an opacity). A partial transparency or partial opacity may result in different volumes or depths of a material receiving different intensities of irradiation. For example, a material at or near the surface facing a light source may receive a higher intensity exposure than material beneath the surface (shaded by partially transparent material above).

FIG. 1 illustrates a pond containing a suspension. Pond 100 typically has sides and a bottom, and is sufficiently deep to contain a suspension 110 at a depth 120. A substance may be characterized by a surface that “faces” a source of light. In FIG. 1, a top surface of the suspension 110 faces light 130 arriving in an incident direction. The top surface of the suspension may be characterized by an area 140. Suspension 110 will often include a liquid 150 and a suspended phase 160.

In some cases, a finite amount of light may be available. For example, an available fluence of sunlight over a given period of time may be the only source of light usable for photosynthesis, and it may be desirable to maximize the conversion of incident sunlight to chemical energy (e.g., using photosynthesis). Uncontrolled exposure may not yield optimal reaction kinetics (e.g., high growth rates, long life, or synthesis of certain chemicals). For example, algae near the top of a pond exposed to intense sunlight may be overexposed. Algae at a substantial depth below the surface may not receive enough sunlight. Predator species may grow preferentially to desired species. Certain conditions may favor the formation of an undesirable chemical over a desirable chemical.

### SUMMARY OF THE INVENTION

Various aspects include exposing a substance to light. A substance may include a suspension comprising a suspended phase and a liquid. In some cases, the suspended phase includes one or more photosynthetic organisms, such as algae, diatoms, and/or bacteria. Exposing may include determining an intensity of the light, determining an opacity of at least a portion of the suspension, and adjusting the opacity in response to the intensity.

In some cases, a response of the suspension is determined. A response may include a measurement of Photosystem performance, photosynthetic rate, fluorescence of certain centers, biomass, lipid mass, photochemical efficiency, photochemical quenching, and the like. Opacity of the suspension may be adjusted in response to the intensity. Portions or regions having different opacities and/or opacity profiles may be created. A concentration of suspended phase in the liquid may be adjusted to control opacity.

A system may comprise a pond configured to contain a suspension. A first inlet may deliver the suspension and/or suspended phase to the pond. A second inlet may deliver liquid to the pond. A sensor may measure light intensity at, within, and/or above the suspension. A sensor may measure depth of the suspension; a sensor may measure the thickness of one or more layers in the suspension.

A system may comprise a pond having a bottom and sides and configured to contain a suspension at a depth and expose the suspension to light having an incident intensity. The suspension may include a suspended phase and a liquid. The suspension may have an opacity to the light that results in at least a first portion of the suspension being characterized by a reduced intensity of the light within the first portion. The reduced intensity may be below a damage threshold associated with the suspended phase. In some cases, the opacity may result in a second portion of the suspension being characterized by an intensity of the light below a recovery threshold characterizing recovery of damage to the suspended phase.

### BRIEF DESCRIPTION OF THE DRAWINGS

FIG. 1 illustrates a pond containing a suspension.

FIG. 2 is a schematic illustration of a variation in light intensity as a function of depth, according to some embodiments.

FIGS. 3A and 3B illustrate two exemplary opacity profiles and their associated intensity profiles for two layered suspensions, according to certain embodiments.

FIG. 4 illustrates an exemplary saturation response, according to some embodiments.

FIG. 5 is a schematic illustration of a capacity to utilize light, according to some embodiments.

FIG. 6 is a schematic illustration of an exemplary damage response.

FIG. 7A is a schematic illustration of a suspension with an opacity profile, according to some embodiments.

FIG. 7B illustrates an intensity profile 750 that may result from an opacity profile as illustrated in FIG. 7A.

FIG. 8 illustrates a method according to some embodiments.

FIG. 9 illustrates a system for exposing a suspension, according to some embodiments.

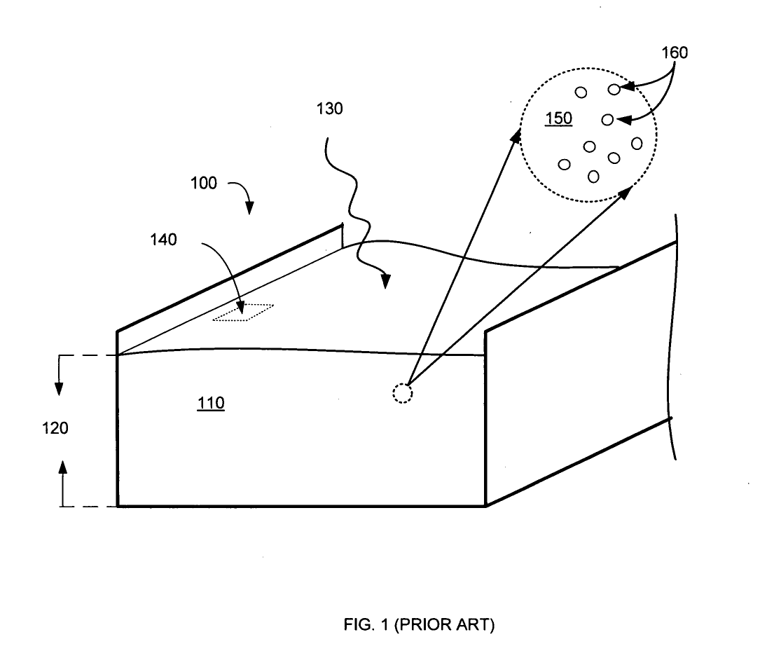

### DETAILED DESCRIPTION OF THE INVENTION

Various aspects provide for optimizing the exposure of a substance to light. Certain aspects include exposing a suspension of photosynthetic organisms to sunlight, and may include optimizing exposure to improve photosynthesis conditions. Optimizing exposure may include maximizing growth rate, maximizing photosynthesis efficiency, maximizing production of a chemical (e.g., a triglyceride), minimizing damage, minimizing predator growth, maximizing a capacity to grow in suboptimal media (e.g., polluted water, brackish water, or water having a pH outside of a preferable range), minimizing requirements for nutrients, and other features.

A suspension may comprise a suspended phase and a liquid. A suspended phase may be a solid, a liquid, a composite, or another phase. In some cases, suspended phases may include small particles (e.g., less than 100 microns, less than 10 microns, less than 1 micron, or even less than 100 nm). A suspension may comprise one or more photosynthetic organisms (e.g., algae, diatoms, bacteria, and the like) suspended in a liquid (e.g., water, seawater, growth media, and the like).

In some cases, optimizing includes maximizing a first property while minimizing a second property. Optimizing may include simultaneously maximizing a plurality of properties. Optimizing may include adjusting various parameters, each of which affects one or more properties, in a way that achieves a desirable level of one or more properties.

Many photosynthetic organisms have a finite capacity to utilize incident light for photosynthesis. A relatively low intensity light may be efficiently utilized (i.e., substantially converted to chemical energy, or converted as efficiently as quantum limits or physiological limits allow). An intense light may “overpower” the organism's photosynthesis capabilities, resulting in a substantial portion of the incident light not being used for photosynthesis. Such unused light may create heat, may pass through the organism, may damage the organism, or may be otherwise lost. In some cases, high intensity light may damage an organism, which may result in decreased photosynthetic efficiency, decreased growth rate, or even death of the organism. Algae growing in water may productively react with sunlight (e.g., to perform photosynthesis) and unproductively react with sunlight (e.g., become damaged). Algae near the surface may receive a high intensity exposure to sunlight. Algae below the surface may receive a lower intensity exposure to sunlight. Some algae may receive too little light to fully utilize their available photosynthetic capacity. Some algae may receive such an intense exposure that they are damaged by the light.

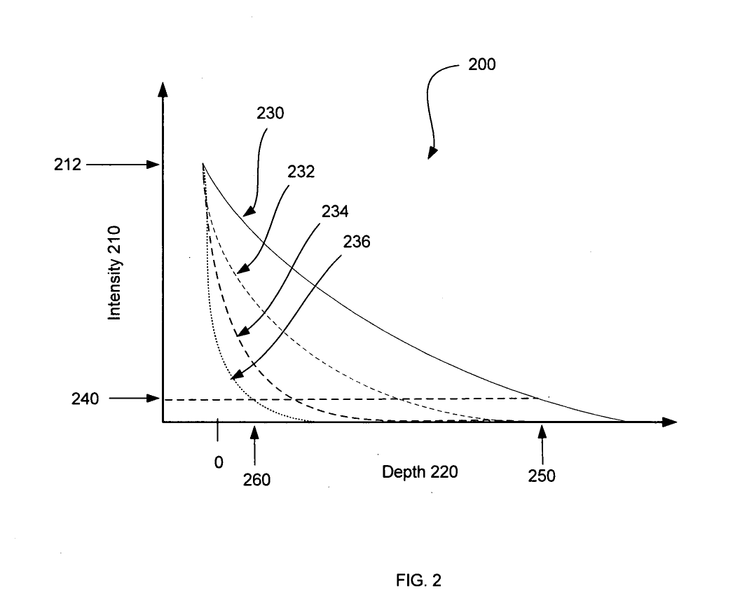

FIG. 2 is a schematic illustration of a variation in light intensity as a function of depth, according to some embodiments. FIG. 2 illustrates several relationships 200 between the measured intensity 210 of light (having an incident intensity 212) as a function of depth 220 beneath a surface of several suspensions. FIG. 2 illustrates several such relationships (i.e., a variation in intensity vs. depth for several different concentrations 230, 232, 234, and 236).

A suspension may be characterized by an opacity to incident light that is small enough that some of the light incident on a given volume is absorbed by the volume and some passes through the volume. As a result, intensity 210 decreases as a function of depth. In some cases (e.g., where a suspension is substantially homogeneous as a function of depth), the slope of the intensity vs. depth function may be steepest near the top and become shallower as depth increases.

Increasing a concentration of suspended phase in the suspension often results in an increased opacity. An increased concentration may result in an increase in the depth dependence of the intensity vs. depth function. For example, suspensions 230, 232, 234, and 236 may have the same liquid and suspended phases, but each may have a different concentration of the suspended phase in the liquid. Suspension 230 may have the most dilute concentration; suspension 232 may be higher concentration than suspension 230; suspension 234 may be higher concentration than suspension 232, and suspension 236 may be higher concentration than suspension 234. As shown in FIG. 2, the depth dependence of intensity may vary dramatically with concentration. In some cases, increasing a concentration may result in a much steeper “drop off” in intensity vs. depth (i.e., a steeper slope near the surface).

Determining relationships among intensity, concentration, and depth (e.g., based on prior knowledge, calculation, modeling, or measurement) may provide for calculating a volume of the suspension exposed to various important intensities—such as an intensity that induces damage, an intensity at which light is optimally harvested, an intensity below which an organism may “recover,” or an intensity so low that photosynthesis cannot sustain growth. For example, FIG. 2 illustrates a photosynthesis limit 240, which in this illustrative example, might correspond to a minimum light intensity needed to maintain the physiological functions of a photosynthetic organism. In this example, a relatively large volume of suspension 230 receives an intensity above photosynthesis limit 240, as reflected by the crossing of the intensity (in suspension 230) below photosynthesis limit 240 at a substantial depth 250 within the suspension. A relatively small volume of suspension 236 receives an intensity above photosynthesis limit 240, as reflected by the crossing of the intensity (in suspension 236) below photosynthesis limit 240 at a smaller depth 260 within the suspension. In some suspensions, opacity may substantially be a function of concentration. In some cases, a similar number of organisms may result in a given decrease in intensity vs. depth, independent of volume (e.g., a shallow, concentrated suspension may absorb as much as a deeper, dilute concentration).

Opacity may vary as a function of position (e.g., as a function of depth). Opacity may vary in different regions in a suspension. In some embodiments, opacity is controlled to change attenuation properties of a region, which may change an intensity of light passing through the region. An opacity profile may characterize opacity as a function of distance in a substance. An opacity profile may result in an intensity profile that is more complex than an intensity profile as shown in FIG. 2 (e.g., having a second derivative that is not constant, does not change linearly, or having different regions requiring characterization by different functions).

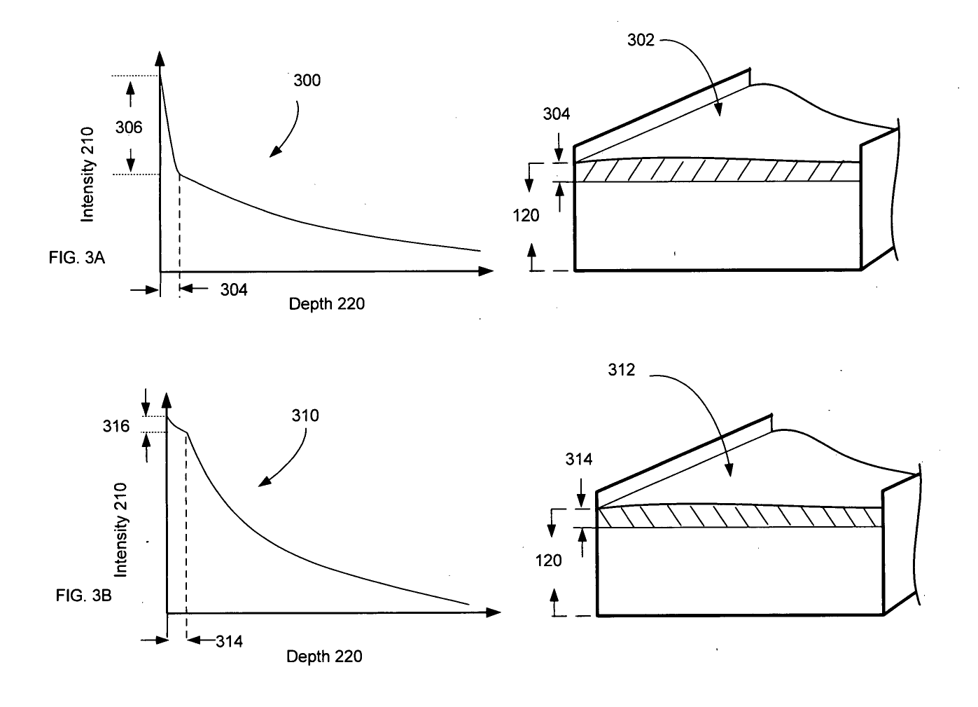

FIGS. 3A and 3B illustrate two exemplary opacity profiles and their associated intensity profiles for two layered suspensions, according to certain embodiments. FIG. 3A illustrates an intensity profile 300 for a layered suspension 302. In this example, layered suspension 302 includes a top layer having a depth 304 beneath the surface. In this example, material in the top layer absorbs, scatters, or otherwise attenuates incident light more than the layer below, resulting in a relatively steep change 306 in intensity 210 over depth 304. Material below the top layer may attenuate light less than the top layer, resulting in a relatively shallower dependence of intensity on depth in the region below the top layer. A layered suspension 302 may be created, for example, by concentrating the suspended phase in the top layer.

FIG. 3B illustrates an intensity profile 310 for a layered suspension 312. In this example, layered suspension 312 includes a top layer having a depth 314 beneath the surface. In this example, material in the top layer absorbs, scatters, or otherwise attenuates incident light less than the region below, resulting in a relatively shallow change 316 in intensity 210 over depth 314. Material below the top layer may attenuate more than the top layer, resulting in a relatively steeper dependence of intensity on depth in the region below the top layer. A layered suspension 312 may be created by diluting the concentration of suspended phase in the top layer. In some cases, adding bubbles to the top layer may increase or decrease attenuation (e.g., depending upon bubble size, bubble concentration, and size/concentration of suspended phase).

Two, three, five, or more layers may be created. Opacity control need not be limited to “layers”—other geometries may be created. Layers need not be entirely discrete; a graded opacity profile (e.g., via a concentration gradient) may be created.

The response of a substance to light may vary with intensity of the light. For example, photosynthetic organisms at the top of a pond may respond to (the relatively intense) light differently than those at the bottom of the pond (responding to less intense light). Certain embodiments feature mixing apparatus, that may circulate or otherwise move portions of the suspension among regions of differing intensities (e.g., from the top of a pond to the bottom).

An intensity profile may be determined (e.g., based on knowledge of a concentration profile and a function describing attenuation vs. concentration). An intensity profile may be measured (e.g., by measuring intensity as a function of distance, such as depth). In some cases, intensity measurement may include incident intensity, diffuse intensity (e.g., light scattered by the surrounding suspension), and/or reflected intensity (e.g, light reflected from the bottom or sides of a pond). Intensity may be measured, for example, using a light meter such as model LI-250A fitted with a Q27723 cosine corrected quantum sensor (LI-COR Biotechnology, Lincoln, Nebr.).

Intensity and/or intensity profiles may be controlled to effect a desired response or range of responses in an irradiated substance. In some cases, a substance may respond linearly to intensity (e.g., a slope of response vs. intensity may be independent of intensity (and nonzero) for some range of intensities). In other cases, a response may be nonlinear (e.g., a response that varies as the square of intensity, the square root of intensity, exp (intensity), log (intensity) and the like).

Certain substances exhibit a saturation response. A saturation response may include a first region (typically at low intensity) in which a first relationship between response and intensity is observed, and a second region (typically at higher intensity) in which a second relationship is observed. Often, the second relationship (at higher intensities) may be associated with a smaller change in response as intensity increases (i.e., the slope decreases at higher intensities).

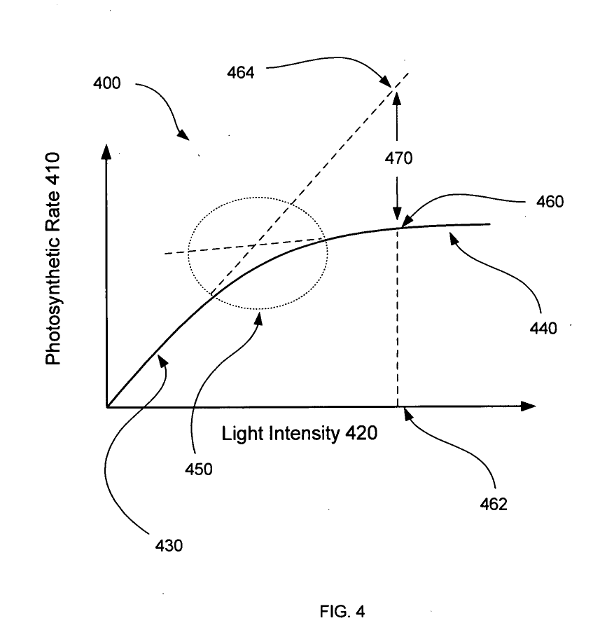

FIG. 4 illustrates an exemplary saturation response, according to some embodiments. FIG. 4 illustrates a schematic response 400 between photosynthetic rate 410 and light intensity 420; such a response may be characteristic of some photosynthetic organisms. Photosynthetic rate 410 may include photosynthetic productivity, photosynthetic efficiency, electron transport rates, lipid productivity, biomass productivity, and the like. Photosynthetic rate 410 may be associated with Photosystem I and/or II production (and may be associated with an electron transport rate associated with Photosystem II).

Response 400 includes a linear regime 430 and a saturation regime 440. Linear regime 430 and saturation regime 440 are separated by a threshold 450. Threshold 450 may be broad or narrow, and may generally be associated with a transition between regimes. For some algae (e.g., of the genus Nannochloropsis), a threshold 450 may be near 200 μEinsteins/m̂2-sec.

Linear regime 430 may be associated with a region of light intensity in which photosynthetic rate 410 is approximately linearly dependent upon light intensity 420. A linear regime 430 may also be characterized as a “light limited” regime, in that productivity is ostensibly limited by the available light. For some organisms, a slope of the productivity vs. intensity response may be associated with a quantum yield of Photosystem II photochemistry.

Saturation regime 440 may be characterized by a productivity below what would be expected based on an extrapolation of the response in linear regime 430 (to higher intensities). For example, an observed photosynthetic rate 460 at intensity 462 may be below an extrapolated photosynthetic rate 464 (based on extrapolating from linear regime 430). An organism receiving an intensity in saturation regime 440 may use a relatively smaller percentage of the incident light for photosynthesis, as compared to an organism in linear regime 430. Such an exposure may overwhelm the photosynthetic capabilities of the organism, resulting in a relatively larger amount of the light not being utilized for photosynthesis. Such an exposure may be characterized by a lost productivity 470, which may be associated with a difference between actual photosynthetic rate and a photosynthetic rate that might be expected based on a productivity response at lower intensities (e.g., in a light limited regime). Certain embodiments include maximizing a number of organisms exposed to an intensity near threshold 450.

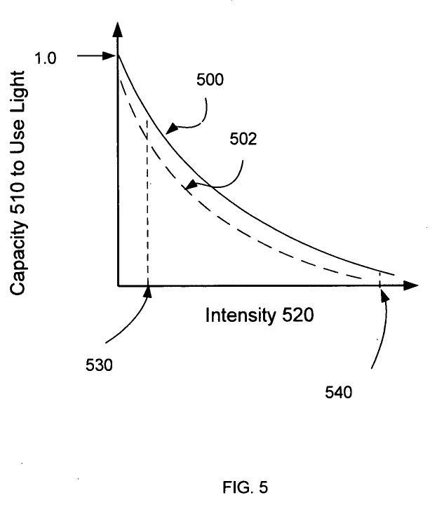

FIG. 5 is a schematic illustration of a capacity to utilize light, according to some embodiments. Many photosynthetic organisms have a finite capacity to use light; fluence beyond this capacity may not be utilized. In some cases, a saturation response in productivity may be associated with an organism's capacity to utilize light.

FIG. 5 illustrates a response 500 characterizing a dependence of capacity 510 to utilize light on intensity 520 of the light. For some organisms, the capacity to utilize light decreases as intensity increases. In the example shown in FIG. 5, the capacity of a substance (e.g., algae) to utilize light is highest at low intensities (normalized to 1.0 at zero intensity) and decreases with increasing intensity. An organism exposed to a low intensity 530 may have a relatively large (e.g., >50%, 70%, or even 90%) capacity to utilize additional light. An organism exposed to a high intensity 540 may have a smaller capacity (e.g., <20%, or even <10%) to utilize additional light. In some embodiments, a parameter that monitors a redox (reduction-oxidation) state of the Photosystem II reaction centers is used to determine capacity 510. In some cases, photochemical quenching of a fluorescence yield (e.g., qP) may be used to determine capacity (e.g., by a ratio of oxidized to reduced reaction centers). For some suspensions, response 500 may “shift” vertically at different concentrations. For example, an algae suspension having a concentration of 50 mg/liter may have a response 500 above the response 502 of a suspension having a concentration of 375 mg/liter.

Overexposure to light may result in inefficient light utilization. Overexposure may damage a substance. For some photosynthetic organisms, exposure to high intensity light may damage photosynthetic apparatus, resulting in an impaired ability to harvest light.

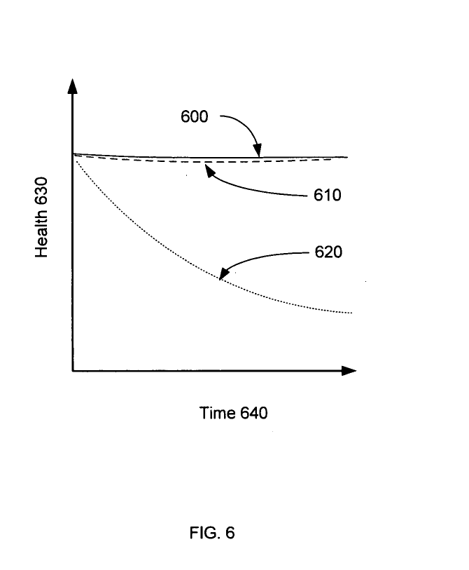

FIG. 6 is a schematic illustration of an exemplary damage response. In some embodiments, damage may be characterized in the inverse (e.g., health); a more damaged organism may be less healthy, less productive, less competitive, and/or less capable of synthesizing a desired chemical (e.g., a lipid). FIG. 6 illustrates three responses 600, 610, and 620 describing population health 630 as a function of time 640 exposed to light. Response 600 may be associated with exposure to relatively low intensity light (e.g., in the light-limited regime or a linear regime 430). Response 610 may be associated with exposure at or near a threshold intensity (e.g., threshold 450). In some embodiments, a damage response may be used to determine a threshold intensity (and/or time). A threshold intensity may be associated with the highest intensity that does not substantially damage an organism exposed for a period of time. Response 620 may describe the health of a population of organisms exposed to an intensity high enough to damage the organisms. In some cases, a damaging intensity may be associated with intensities in saturation regime 440 (e.g., intensity 462).

Certain embodiments include a wavelength dependence in intensity calculations or measurements. For example, a low intensity of a shorter wavelength (e.g., ultraviolet) may damage more than a higher intensity of a longer wavelength (e.g., visible). For some light sources, a range of wavelengths may be simultaneously incident (e.g., UV to IR). For light sources such as sunlight, a wavelength distribution may change during the day, with the relative intensities (e.g., of UV vs. visible) changing from morning to midday to evening.

Some embodiments include the determination of population health using a measurement of damage (e.g., to the suspended phase or to the liquid). In some cases, a slope associated with a relationship between Photosystem II and intensity may be used to determine damage (e.g., a smaller slope may be indicative of damage). Certain embodiments include determining damage by assessing an integrated exposure time to an integrated intensity (e.g., an historical exposure). Some embodiments include determining damage via a measurement of a photoinhibition fluorescence parameter (e.g., measuring Fv/Fm, a ratio of variable to maximum chlorophyll fluorescence).

Some organisms may at least partially recover from damage (e.g., by spending time in a “dark” zone). Some photosynthetic organisms may recover with a Photosystem II repair cycle. At some intensities, a population of organisms may repair damage as fast as damage occurs, essentially “keeping up” with the damage. At higher intensities, a population may be damaged faster than its ability to repair itself, resulting in an increase in average damage over time.

Certain embodiments include exposing organisms to a region having high intensity light (e.g., wherein they shade organisms below). In such a case, a “sunscreen” or “shading” region may reduce an intensity of light passing to organisms below. In some cases, suspensions are mixed such that a first portion of the suspension shades the organisms below for some period of time, but are not exposed for a long enough time to cause irrecoverable damage. Certain embodiments provide for transporting (e.g., via mixing) organisms from a region having a damaging light intensity to a region having an intensity under which damaged organisms may recover.

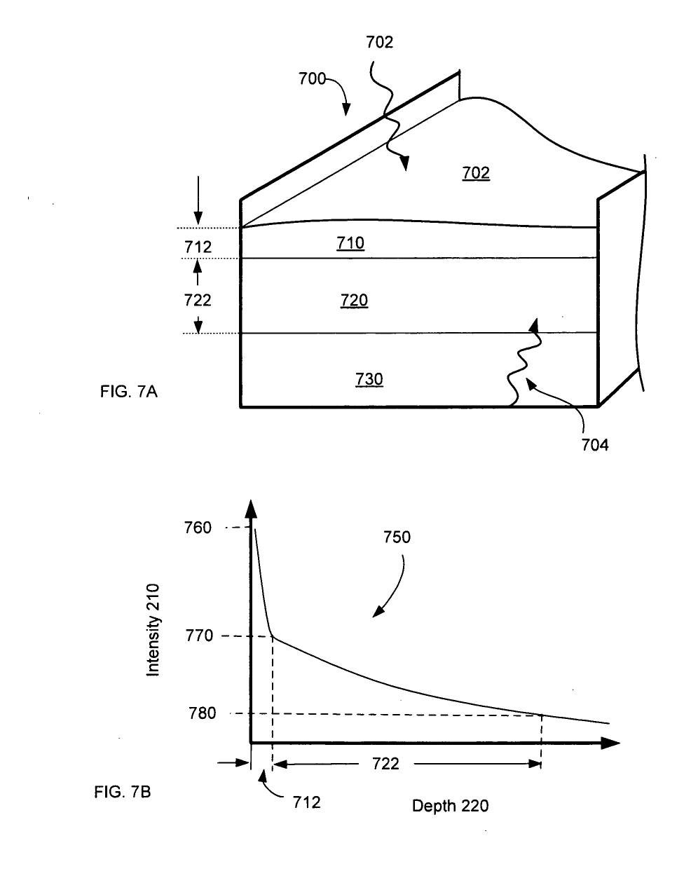

FIG. 7A is a schematic illustration of a suspension with an opacity profile, according to some embodiments. For illustrative purposes, the opacity profile may be described in terms an intensity profile, which may describe the effect of each region on incident intensity 702 and reflected intensity 704. Portions of suspension 702 in this example include shading portion 710, efficient portion 720, and optional recovery portion 730. Shading portion 710 has a thickness 712; efficient portion 720 has a thickness 722.

FIG. 7B illustrates an intensity profile 750 that may result from portions disposed as illustrated in FIG. 7A. In this example, incident intensity 760 may be larger than the capacity of the algae to efficiently utilize the light, and may be large enough to damage the algae (e.g., midday sunlight near the equator). Threshold intensity 770 may be associated with an intensity at or below which algae are not damaged, or an intensity at or below which algae efficiently utilize light. Optional recovery intensity 780 is associated with an intensity below which algae may recover from damage (e.g., from high intensity exposure).

Shading portion 710 may be disposed at the top of suspension 130. In some embodiments, a concentration of a suspended phase (e.g., suspended algae or diatoms) in shading portion 710 may be chosen such that shading portion 710 attenuates a portion of incident light 702, such that regions below shading portion 710 are exposed to intensities below a damage intensity, or even below a recovery intensity 780. In some embodiments, bubbles or other sources of diffusion or scattering of incident light may be introduced (e.g., into shading portion 710 or efficient portion 720) to attenuate or disperse light. Thickness 712 may be large enough to “shave off” damaging intensities, but small enough to allow passage of “usable light” to organisms below.

Algae may be cycled through shading portion 710 and other portions, such that damage that might be incurred due to exposure in shading portion 710 may be healed during a subsequent time period in a region of lower intensity. A ratio of time spent in shading portion 710 vs. time spent in other portions may be determined by damage kinetics at intensity 760 and recovery kinetics (at damaging, efficient, and/or recovery intensities). For intensities resulting in greater damage, algae may spend less time in shading portion 710. For some intensities, a “sacrificial layer” of algae, diatoms, or other organisms may bear the brunt of intensity attenuation.

Thickness 722 of efficient portion 720 may be chosen to maximize the number of algae exposed to an intensity that they have the capacity to harvest. Thicknesses may be chosen to minimize the number of algae exposed to an intensity above that which they have the capacity to harvest. Mixing within efficient portion 720 may be used to cycle algae from lower to higher intensities. Depending upon damage kinetics, recovery/healing kinetics, and incident intensity, algae may be cycled into recovery portion 730 for sufficient time to recover a lost ability to harvest light.

An opacity profile may characterize a change in opacity as a function of distance (e.g., depth). Portions of different opacity characteristics may create an intensity profile more complex than that of a homogeneous suspension. In some cases, an opacity profile may include a relatively abrupt change in the slope of opacity vs. concentration (e.g., a large change in the second derivative of a function describing intensity vs. depth). An opacity profile associated with a pond may be changed during the course of the day (e.g., morning to midday to evening), pursuant to weather changes (sunny, cloudy, raining), and/or according to seasonal changes. An opacity profile may be changed according to geographical location (e.g., near the Equator, between the Tropics, outside the tropics). In some embodiments, evaporation of a liquid may be used to concentrate algae near the surface, which may cause increased concentration as incident radiation increases. An opacity profile may be adjusted in response to an angle of incidence of the light. For example, a shading portion might be removed for low angle light (morning or evening) and created or increased when the sun is overhead. Certain embodiments include sensors that track the sun.

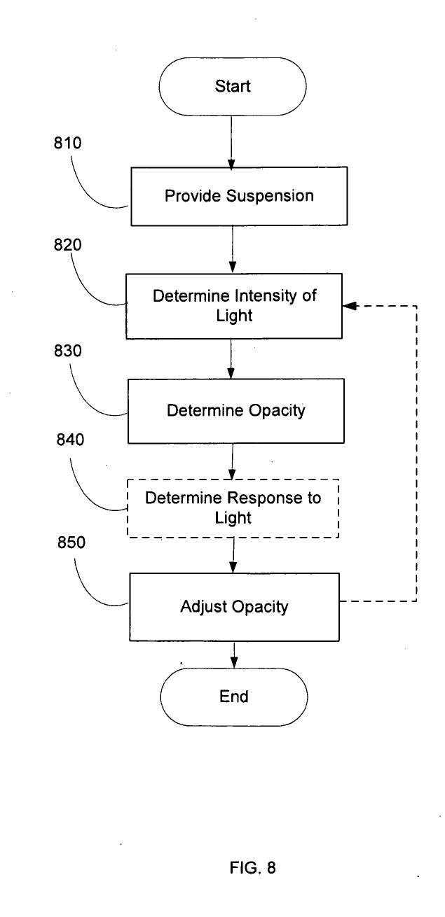

FIG. 8 illustrates a method according to some embodiments. A suspension is provided in step 810. A suspension may comprise a plurality of photosynthetic organisms suspended in a liquid (e.g., water, seawater, brackish water, and the like). In step 820 an intensity of light may be determined. Intensity may be determined by measuring the light. Incident light may be measured; scattered or diffuse light may be measured. In some embodiments, intensity may be determined outside the suspension (e.g., an intensity of light incident on the suspension). Determining intensity may include determining an incident angle associated with incident light. In some embodiments, intensity is determined at one or more points in the suspension. Intensity may be determined by calculation (e.g., based on weather, time of day, season, and the like). Intensity may be determined in concert with a determination of opacity. Determining intensity may include determining a depth dependence of intensity. Determining may include determining the intensity of reflected light (e.g., from the bottom). Determining intensity may include determining intensity over a period of time, and may include determining an average intensity, a minimum intensity, a maximum intensity, an integrated intensity over the period of time, and/or a temporal dependence of intensity.

Opacity of the suspension may be determined in step 830. Determining the opacity may include determining an opacity profile. Determining the opacity may include calculating the opacity (e.g., based on concentration). Determining the opacity may include the determination of light intensity (e.g., by measuring intensity and calculating the resulting opacity). Opacity may be measured (e.g., with a submerged sensor having a known optical path and light source). In some embodiments, opacity is determined by measuring intensity within the suspension. Opacity may be measured at a plurality of points in the suspension (e.g., a plurality of depths). Opacity may be determined as a function of the determined intensity.

In optional step 840, a response of the suspension to the light may be determined. In some cases, a determination of intensity (e.g., incident sunlight) may provide sufficient information to determine a need for opacity adjustment and (by extension) adjust opacity. In some cases, a response of the suspension (e.g., to the current or recent intensity) may be determined. Determining a response may include calculating, estimating, and/or looking up an expected response. Determining a response may include measuring the response. A response may be determined for one or more portions of the suspension (e.g., organisms near the bottom, organisms near the top, organisms in the middle). Determining a response may include determining a plurality of responses (e.g., at different intensities through the depth). Determining a response may include determining an average response, a mean response, a minimum response, a maximum response, and/or a response associated with a particular transition (e.g., an intensity threshold). Determining a response may include determining an integrated response over a period of time (e.g., over an integrated intensity over a period of time). A response may include a property of the organisms in the suspension. Determining a response may include measuring photochemical rate, photoinhibition, response of Photosystem I and/or II, Fv/Fm, photochemical quenching, concentration, biomass, amount of lipids, amount of carbohydrates, and/or amount of protein. Biomass production, chemical production, population health, and other responses may be determined (e.g., measured). The productivity of a preferred chemical (e.g., a lipid) may be measured. A concentration of a predator may be measured. A change in opacity and/or opacity profile may be measured. A salinity may be measured. A response may include a property of the liquid. A response may include a change in temperature of the suspension.

Opacity may be adjusted in step 850. Opacity may be adjusted to optimize exposure of the suspension to the light. Opacity may be adjusted to maximize a growth rate of organisms in the suspension. Opacity may be adjusted to maximize a production rate of certain chemicals (e.g., lipids, omega3 or omega6 fatty acids, sugars, and/or other components). Opacity may be adjusted to minimize damage to the suspension. Opacity may be adjusted to minimize growth of a competitive species or a predator. Opacity may be adjusted by changing the depth of the suspension (e.g., while keeping concentration constant). Opacity may be adjusted by changing the concentration of the suspended phase in the liquid. Opacity may be adjusted by changing a reflectance of a surface (e.g., moving the suspension to a region of the pond having bottom and/or sides with different reflectivity or albedo). Opacity may be adjusted in response to changing light intensity (e.g., decreasing opacity in low light conditions). Opacity may be adjusted in response to temperature changes (e.g., decreasing opacity at cool temperatures), and/or weather effects (e.g., rain, evaporation). In some situations (e.g., where intensity of light is determined at one or more points within the suspension), an adjustment in opacity of the suspension may be combined with a determination of intensity (e.g., at one or more points within the “adjusted-opacity” suspension). For example, an expected response to a determined intensity may cause an opacity adjustment. The opacity adjustment may be expected to change the opacity profile through the suspension, which might be expected to cause a change in light intensity at one or more points within the suspension. Intensity might be measured in the adjusted suspension to compare actual and adjusted intensities. Some substances (e.g., suspensions of algae or diatoms in water) may be moving, flowing, circulating, or otherwise in motion during exposure to light. A flow pattern may characterize flow within the suspension, and may characterize circulations, velocities, residence times at various points, changes in concentration, changes in shape (e.g., depth of a pond), and other characteristics.

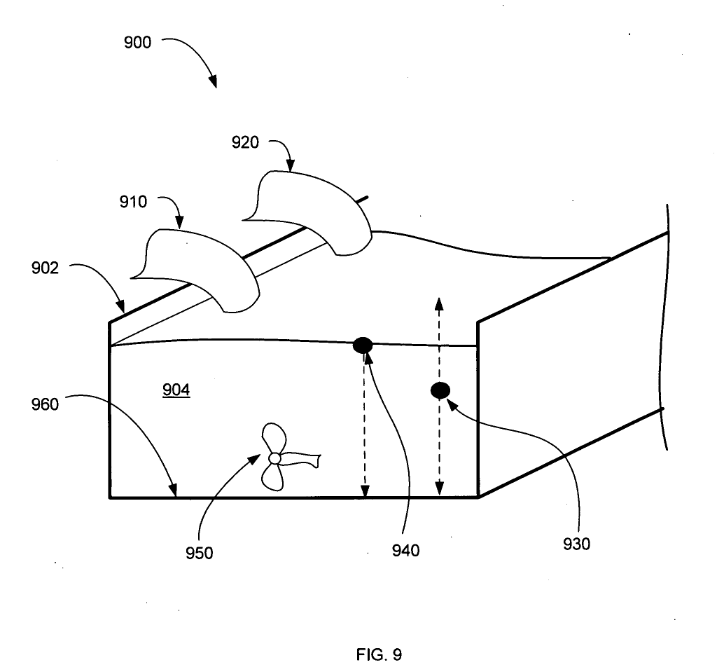

FIG. 9 illustrates a system for exposing a suspension, according to some embodiments. System 900 includes a pond 902 configured to contain suspension 904. A first inlet 910 may deliver suspension 904 or a suspended phase to pond 902. An optional second inlet 920 may deliver liquid to pond 902. Optional outlets for any of the suspension, liquid, and suspended phase are not shown. System 900 may include a sensor 930 (e.g., to measure light intensity, opacity, concentration, or other parameters). In some embodiments, sensor 930 may be disposed at different locations within or above suspension 904. Sensor 930 may sense incident light, diffuse light, light reflected from a side or bottom of pond 902, and/or light scattered by suspension 904. Additional sensors (e.g., temperature) may be included. An optional depth sensor 940 may be included. Depth sensor 940 may measure the depth of suspension 904. One or more sensors configured to measure thicknesses of different layers, portions, or regions of suspension 904 may be included (e.g., optical sensors). In some cases, a plurality of optical sensors is vertically disposed along a wall of pond 902. Optional mixing means 950 may be included. Mixing means may include a propeller, a jet, a paddle, a blade, and/or other means for stirring or otherwise causing convection in suspension 904 and/or a portion of suspension 904. In some embodiments, a plurality of mixing means is disposed throughout suspension 904. Pond 902 may include an inner surface 960 whose reflectivity is controlled. In some cases, a substantially black inner surface 960 may be implemented. In some cases, a substantially white and/or silver colored inner surface 960 may be implemented.

Various embodiments provide for increasing a productivity of a suspension comprising photosynthetic organisms. Suspension parameters may be adjusted in response to changes in incident radiation, and may be adjusted to optimize one or more properties of the suspension.

Some embodiments include sensors to sense various parameters (e.g., concentration, depth, health, photosynthetic rate, clarity, pH, mass, transparency, opacity, light intensity, fluorescence, qP, Fv/Fm, and other characteristics). Apparatus may monitor various sensors, and systems may be actuated by automated controls (solenoid, pneumatic, piezoelectric, and the like). Some embodiments include a computer readable storage medium coupled to a processor and memory. Executable instructions stored on the computer readable storage medium may be executed by the processor to perform various methods described herein. Sensors and actuators may be coupled to the processor, providing input and receiving instructions associated with various methods. Certain instructions provide for closed-loop control of various parameters via coupled sensors providing input and coupled actuators receiving instructions to adjust parameters.

The above description is illustrative and not restrictive. Many variations of the invention will become apparent to those of skill in the art upon review of this disclosure. The scope of the invention should, therefore, be determined not with reference to the above description, but instead should be determined with reference to the appended claims along with their full scope of equivalents.

What is claimed is:

1. A method for exposing a suspension to light, the method comprising:
determining an intensity of the light;
determining an opacity of at least a portion of the suspension; and
adjusting the opacity in response to the intensity.

2. The method of claim 1, wherein the suspension includes a liquid and a plurality of photosynthetic organisms.

3. The method of claim 2, wherein adjusting the opacity includes adjusting a concentration of the organisms in the liquid.

4. The method of claim 3, wherein adjusting the concentration includes diluting the suspension.

5. The method of claim 2, wherein adjusting the opacity includes evaporating at least a portion of the liquid.

6. The method of claim 1, wherein adjusting the opacity includes altering a flow pattern associated with the suspension.

7. The method of claim 2, wherein adjusting the opacity includes segregating the suspension into regions having different concentrations of the organisms in the liquid.

8. The method of claim 7, wherein the segregated suspension includes a top region having a higher concentration than a bottom region having a lower concentration.

9. The method of claim 2, wherein the suspension includes one or more diatoms.

10. The method of claim 2, wherein the suspension includes one or more algae.

11. The method of claim 10, wherein any of the algae includes a member of the genus Nannochloropsis.

12. The method of claim 2, further comprising determining a property of the organisms.

13. The method of claim 12, wherein the property includes a response to the light.

14. The method of claim 13, wherein the property includes an integrated response to the light over a period of time during which the suspension was exposed to the light.

15. The method of claim 12, wherein the property includes a photosynthetic efficiency of the organisms.

16. The method of claim 12, wherein the property is associated with a Photosystem II response.

17. The method of claim 12, wherein the property includes a capacity of the organisms to perform photosynthesis.

18. The method of claim 12, wherein the property includes a photochemical quenching characteristic of the organisms.

19. The method of claim 18, wherein the property is associated with a Photosystem I response.

20. The method of claim 12, wherein the property includes a damage parameter associated with damage to the organisms.

21. The method of claim 20, wherein the damage at least partially results from an exposure to the light.

22. The method of claim 20, wherein the property includes a photoinhibition response.

23. The method of claim 12, wherein determining the property includes sampling a plurality of points within the suspension.

24. The method of claim 23, wherein two or more points in the plurality are characterized by different intensities of exposure to the light.

25. The method of claim 2, wherein the adjusted opacity maximizes an exposure of the organisms to an intensity corresponding to an efficiency threshold.

26. The method of claim 2, wherein the adjusted opacity minimizes an exposure of the organisms to an intensity above a damage threshold.

27. The method of claim 1, wherein adjusting the opacity includes adjusting a distance between a top and a bottom of the suspension.

28. The method of claim 1, wherein the suspension is characterized by a concentration of a suspended phase in a liquid, and a first concentration prior to adjusting the opacity is different than a second concentration after adjusting the opacity.

29. The method of claim 1, wherein determining the intensity includes measuring the intensity.

30. The method of claim 29, wherein the intensity is measured at one or more points within the suspension.

31. The method of claim 29, wherein the measured intensity includes an incident intensity.

32. The method of claim 29, wherein the measured intensity includes a reflected intensity.

33. The method of claim 32, wherein the reflected intensity includes reflection from the bottom.

34. The method of claim 1, wherein the suspension comprises a liquid and a suspended phase, and determining the opacity includes:
determining a concentration of the suspended phase in the liquid; and
calculating the opacity based on the concentration.

35. The method of claim 1, wherein determining the opacity includes measuring the opacity.

36. The method of claim 1, wherein the determining the opacity includes determining the opacity at a plurality of points within the suspension.

37. The method of claim 1, wherein determining the opacity includes determining an opacity profile in a first direction.

38. The method of claim 37, wherein the first direction is within 45 degrees of an incident direction associated with the light.

39. The method of claim 37, wherein the first direction is within 45 degrees of a reflected direction associated with a reflection of the light from a bottom of the suspension.

40. The method of claim 1, wherein determining the intensity includes measuring the intensity.

41. A system comprising:
a pond configured to contain a suspension at a depth, the suspension comprising a suspended phase and a liquid;
a first inlet configured to deliver the suspension to the pond; and
a sensor to measure an intensity of light within the pond.

42. The system of claim 41, further comprising a second inlet to deliver the liquid to the pond.

43. The system of claim 41, wherein the sensor is configured to measure an incident intensity of the light.

44. The system of claim 41, wherein the sensor is configured to measure a reflected intensity resulting from a reflection of the light from a bottom or side of the pond.

45. The system of claim 41, further comprising a depth gauge configured to measure a distance between a bottom of the pond and a top surface of the suspension.

46. The system of claim 41, wherein the sensor is disposed within the delivered suspension.

47. A system comprising:
a pond having a bottom and sides and configured to contain a suspension at a depth and expose the suspension to light having an incident intensity, the suspension comprising a suspended phase and a liquid, the suspension having an opacity to the light that results in at least a first portion of the suspension being characterized by a reduced intensity of the light within the first portion, the reduced intensity below a damage threshold associated with the suspended phase.

48. The system of claim 47, wherein the opacity results in at least a second portion of the suspension being characterized by a recovery intensity of the light within the second portion, the recovery intensity below a recovery threshold associated with the suspended phase.
